(19) United States
(12) Patent Application Publication
(10) Pub. No.: US 2025/0360061 A1
(43) Pub. Date: Nov. 27, 2025

(54) FOAM CONDITIONING COMPOSITION

(71) Applicant: Church & Dwight Co., Inc., Princeton, NJ (US)

(72) Inventors: Gabrielle BARTON, Folkestone, Kent (GB); Alexander RICHARDSON, Chatham (GB); Shirish DESALE, Ashford, Kent (GB); Maria DE MIQUEL, Folkestone, Kent (GB)

(73) Assignee: Church & Dwight Co., Inc., Princeton, NJ (US)

(21) Appl. No.: 18/873,598

(22) PCT Filed: Jun. 13, 2023
(86) PCT No.: PCT/IB2023/056102

§ 371 Date: Dec. 10, 2024

Related U.S. Application Data
(60) Provisional application No. 63/352,469, filed on Jun. 15, 2022.

(51) Int. Cl. A61K 8/04; A61K 8/34; A61K 8/891; A61Q 5/12

(52) U.S. Cl. CPC A61K 8/046; A61K 8/345; A61K 8/891; A61Q 5/12

(57) ABSTRACT

The present disclosure provides conditioning compositions effective for imparting one or more conditioning effects when applied to substantially dry hair. The conditioning composition is delivered from a container to be used in the form of a stable foam. The conditioning composition can include: an aqueous liquid package; a gaseous package effective as a propellant; a conditioning package comprising a plurality of conditioning agents present in amounts effective to be retained by hair to which the conditioning composition is applied and to impart to the hair at least one conditioning effect; and a foaming package comprising a plurality of components effective provide the conditioning composition as a stable foam upon dispensing of the conditioning composition from a container.

### FIELD OF THE INVENTION

The present disclosure relates to compositions for application to hair for providing conditioning effects, particularly for application to dry hair, and particularly to provide conditioning with rapid drying and without requirement for rinsing away with water.

### BACKGROUND

Dry hair cleaning compositions for the rapid cleansing of hair are well known and can be used on dry hair in order to freshen up the hair and/or to remove excess oils and other undesirables found on dirty hair. These compositions are particularly useful when time is short and one cannot wash the hair using wet shampoo. Dry shampoo compositions can be formulated to provide conditioning effects. As the primary purpose of a dry shampoo is for removal of grease and oil from the hair, however, it can be difficult to achieve both cleansing and conditioning in the same formulation with optimum effects for both cleansing and conditioning. Previous efforts to rectify this problem include use of leave-in conditioners, and some formulations have been proposed for application to dry hair as well as wet hair. Known conditioning compositions for addition to dry hair can leave the hair feeling “heavy”, can reduce the volume that is desirably present in freshly cleaned hair, and can reduce the benefits of previous hair cleansing by leaving films or other residue that leaves the hair feeling less clean. Previously proposed conditioning compositions for use on dry hair can leave the hair feeling wet for an extended time, and this can effectively reverse the benefit of using “dry” shampoos and/or “dry” conditioners—i.e., the ability to apply and then quickly move to other tasks without time-consuming drying of the hair or just proceeding with wet hair for an undesired length of time.

Accordingly, there is still a desire and a need to provide hair treating compositions that are suitable for application to dry hair and that will provide lasting conditioning without the need to rinse away and without leaving the hair with a feeling of “heaviness” or “oiliness”. In particular, there is a need to provide a foaming product that, without requiring rinsing, has the desired conditioning performance of compositions that typically do require rinsing. There also is a need to provide such products in a form that can be applied to dry hair while imparting only minimal wetting that enables the hair to rapidly return to substantial dryness after application.

### SUMMARY OF THE INVENTION

The present disclosure relates to hair care compositions. The compositions particularly are useful for conditioning hair and are further particularly useful for conditioning dry hair (i.e., hair that has not previously been wetted with added water, such as in a shower or bath). The conditioning compositions are configured to impart to hair at least one conditioning effect, and the compositions are configured to do so without the requirement for added water to disperse the conditioning composition through the hair and without the requirement for added water to rinse away the conditioning composition. The conditioning composition is thus a leave-in conditioning composition that achieves the desired conditioning effects without undesired effects, such as leaving hair appearing wet, heavy, oily, limp, or other effects that are often associated with typical leave-in conditioners.

The present conditioning compositions achieve the desired conditioning effects by including all of aqueous liquid package components, gaseous package components, conditioning package components, and foaming package components. The components of each package provide a specific function as further described herein so that the conditioning composition is delivered from a container as a stable foam that enables excellent dispersal of relatively high amounts of conditioning agents and/or a relatively high number of conditioning agents. Increased conditioning agents are enabled for delivery in a foam in light of the combination of foaming package components and the relative amounts of the aqueous liquid package components and the gaseous package components that lead to formation of a foam with increased stability at the liquid film network surrounding the gas bubbles or pockets in the foam. The foam is thus stable for a time sufficient to allow a user to process the foam through as much of the hair as desired, up to the full head of hair, and the foam stably retains high dispersion of the conditioning agents throughout the foam in the form of small droplets so that the conditioning agents in particularly are evenly distributed throughout the hair while avoiding patchy application, which that can lead to an oily appearance to parts of the hair, and which does not achieve the desired conditioning effects to substantially all of the hair to which the composition is applied.

In one or more embodiments, the present disclosure thus can provide conditioning compositions comprising: an aqueous liquid package; a gaseous package effective as a propellant; a conditioning package comprising a plurality of conditioning agents present in amounts effective to be retained by hair to which the conditioning composition is applied and to impart to the hair at least one conditioning effect; and a foaming package comprising a plurality of components effective provide the conditioning composition as a stable foam upon dispensing of the conditioning composition from a container. The conditioning compositions can be further defined in relation to any one or more of the following statements, which statements can be combined in any number and order. Further, the following statements may themselves be further defined in relation to additional disclosure that follows herein, and the compositions should not be artificially limited to solely the statements themselves unless the disclosure expressly indicates that such limitation is desired.

The aqueous liquid package can comprise water and an alcohol present in a ratio of about 12 to 1 to a ratio of about 6 to 1 on a weight basis.

The plurality of conditioning agents in the conditioning package can comprise at least a hydrating component effective to impart to the hair a hydrating effect and at least a smoothing component effective to impart to the hair a smoothing effect.

The hydrating component can comprise a polyol, and the smoothing component comprises a silicone.

The polyol can be present in an amount of at least 2.5% by weight based on the overall weight of the conditioning composition, and the silicone is present in an amount of at least 4% by weight based on the total weight of the composition.

The polyol can be glycerin, and the silicone can be PEG-8 dimethicone.

The foaming package can comprise each of a foaming agent, a foam preservative, and a foam stabilizer.

The foaming agent can be a water soluble liquid, the foam preservative can be an oil, and the foam stabilizer can be a particulate solid.

The particulate solid foam stabilizer can be effective to increase smoothness of the foam, increase creaminess of the foam, and slow disintegration of the foam.

The foaming package can include particulate starch in an amount substantially ineffective for absorbing oil from hair.

The particulate starch can be rice starch present in an amount of no greater than 4.5% by weight based on the overall weight of the conditioning composition.

The aqueous liquid package can comprise about 60% to about 75% by weight of the composition, the gaseous package can comprise about 16% to about 24% by weight of the composition, the conditioning package can comprise about 5% to about 10% by weight of the composition, and the foaming package can comprise about 3% to about 7% by weight of the composition, each of the foregoing being based on the total weight of the composition.

The conditioning composition further can comprise at least one of a fragrance, a plant extract, and a temporary dye.

The stable foam provided upon dispensing of the conditioning composition from container can have a density of at least 0.3 g/cm³.

The stable foam provided upon dispensing of the conditioning composition from container can an initial volume, and a retained volume of the stable foam at a time of 1 minute after dispensing can be at least 90% of the initial volume.

The composition can be effective for conditioning the hair such that the at least one conditioning effect imparted to the hair is retained for at least 8 hours after the conditioning composition is applied to the hair.

The present disclosure, in some embodiments, further can provide methods of conditioning hair. For example, such methods can comprise applying to the hair when the hair is substantially dry a conditioning composition comprising: an aqueous liquid package; a gaseous package effective as a propellant; a conditioning package comprising a plurality of conditioning agents present in amounts effective to be retained by hair to which the conditioning composition is applied and to impart to the hair at least one conditioning effect; and a foaming package comprising a plurality of components effective provide the conditioning composition as a stable foam upon dispensing of the conditioning composition from a container. The conditioning methods can be further defined in relation to any one or more of the following statements, which statements can be combined in any number and order. Further, the following statements may themselves be further defined in relation to additional disclosure that follows herein, and the methods should not be artificially limited to solely the statements themselves unless the disclosure expressly indicates that such limitation is desired

Applying the conditioning composition can be preceded by cleaning the hair with a dry shampoo composition.

Applying the conditioning composition can be effective to impart to the hair the at least one conditioning effect in the substantial absence of providing any cleaning effect to the hair.

### BRIEF DESCRIPTION OF THE FIGURES

FIGS. 1 through 6 are photographs of three foams produced by dispensing three different compositions from separate containers, wherein the compositions differ in relation to presence of a particulate solid foam stabilizer and amount of the stabilizer present, and wherein the photographs were taken at the time of dispensing and at different times thereafter to illustrate the timing and progression of foam dissipation relative to the presence of the particulate solid foam stabilizer and the amount of the particulate solid foam stabilizer included in the composition.

### DETAILED DESCRIPTION OF THE INVENTION

The present disclosure now will be described more fully hereinafter with reference to the accompanying drawings. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

The present disclosure provides conditioning compositions that are configured for imparting one or more conditioning effects to hair to which the compositions are applied. The conditioning compositions are configured for use with and application to hair that is substantially dry. The conditioning compositions are likewise configured for being left in the hair after application and, as such, the compositions are configured as leave-in conditioning compositions. The conditioning compositions thus are not rinsed from hair after application and do not require rinsing away with water in order to avoid leaving the hair with an oily or greasy feel or appearance or in order to avoid leaving the hair appearing wet or feely artificially heavy relative to hair without the conditioning composition applied thereto. In this regard, hair that is substantially dry prior to addition of the conditioning composition is understood to indicate that the hair has not been wetted by addition of water or other moisture separate from water or other moisture that is naturally retained by the hair itself. Hair naturally retains some level of moisture even hours after any externally applied water has been removed by external means (e.g., toweling dry or blow drying) or by natural evaporation. Substantially dry hair as used herein thus indicates that any moisture present is naturally present in the hair structure and is not externally applied water or other moisture.

The conditioning composition, as further described herein, includes aqueous components, and application of the conditioning composition to hair will initially add a sensation of wetness to the hair. The compositions are configured, however, so that the wetness rapidly departs from the hair, leaving the hair with a feeling that is dry in that no externally applied water is present but also with a feeling of being hydrated in that the hair itself has taken up the moisturizing and conditioning components of the conditioning composition applied thereto.

The conditioning composition is configured to be dispensed from a container in the form of a foam or an aerosol foam. As such, the conditioning components of the conditioning composition are widely dispersed by intervening gas pockets, gas bubbles, or other gaseous phase components. This foam structure achieved through a specific combination of components as otherwise described herein enables the delivery of multiple conditioning components in sufficient quantities to impart conditioning effects and conditioning efficacy not heretofore seen in compositions intended for application to dry hair. Consumer testing described later herein validated the improved conditioning effects provided according to the present disclosure, and this conditioning effect is believed to be achieved only according to a specific combination of components that achieve a stable foam that is high in conditioning components.

In various embodiments, conditioning compositions according to the present disclosure can comprise four separate “packages” or groups of components, each package being configured to impart specific qualities and effects to the overall conditioning composition, and the packages interacting to provide the conditioning composition in the form of a foam that is stable and this is also casily applied with hands to hair so that the conditioning effects are imparted to the hair and so that the hair rapidly returns to a substantially dry condition while retaining the imparted conditioning effects for a prolonged period of time. The compositions in particular can comprise an aqueous liquid package, a gaseous package, a conditioning package, and a foaming package.

An aqueous liquid package for forming the present conditioning composition preferably comprises a sufficient amount of material to function as a solvent for the water soluble components of the composition and to function as a carrier for water insoluble components. The liquid phase package comprises at least water but preferably comprises at least one additional component. Water can be present in an amount of about 40% to about 80%, about 45% to about 75%, or about 50% to about 70% by weight based on the total weight of the conditioning composition. The at least one additional component particularly can be at least one alcohol. Ethanol is a non-limiting example of a suitable alcohol. In some embodiments, the alcohol can be a denatured ethanol, such as ethanol denatured with tert-butyl alcohol and denatonium benzoate (or other suitable denaturant). The alcohol component can be present in amounts of about 4% to about 10%, about 5% to about 9%, or about 6% to about 8% by weight based on the total weight of the conditioning composition. In some embodiments it can be beneficial for the water and the alcohol to be present in the composition in specific ratios. For example, a ratio of a concentration of the water in the conditioning composition to a concentration of the alcohol in the conditioning composition can be about 12 to 1 (water to alcohol) to about 6 to 1 (water to alcohol). In further embodiments, the ratio can be about 10 to 1 to about 7 to 1, or about 9 to 1 to about 8 to 1. The aqueous liquid package as a whole can comprise about 55% to about 80%, about 60% to about 75%, or about 65% to about 70% by weight of the conditioning composition.

A gaseous package for use in the present conditioning compositions can be one or more gases that are effective as a propellant. Because the conditioning composition is configured to be delivered for use as a foam, the gaseous package should be effective to propel the composition from the container in a manner that ensure proper foam formation. Because the present composition is configured as a conditioning composition, the composition can include a greater number of conditioning components and/or a greater concentration of conditioning components that are realistically possible in known compositions. The greater number and/or greater concentration of conditioning components can have the undesired effect of causing the delivered foam to be overly heavy and thus prematurely collapse before application to the hair. The greater number and/or greater concentration of conditioning components likewise can have the undesired effect of causing the delivered foam to feel oily in the hand due to incomplete dispersal of the conditioning components throughout the foam. Such incomplete dispersal of conditioning components can also hamper even application of the conditioning component itself in the hair. For example, incomplete dispersal can lead to “pockets” of conditioning component in small areas of the hair (causing these areas to feel oily and/or heavy) and exclusion of conditioning component from other areas of the hair (preventing these areas from obtaining the desired conditioning effects). The disclosed conditioning composition overcomes such problems though proper balancing of the various packages of components, through proper combination of foaming package components, and through proper use of the gaseous propellant(s).

The gaseous package, in particular, can address the potential pitfalls of increased conditioning component content in the composition by ensuring that the foam that forms upon dispensing from the container has the necessary volume so that the conditioning components are substantially evenly dispersed throughout the foam despite the much heavier weight and density of the conditioning components compared to the components of the aqueous liquid package. This even dispersal is enhanced by the function of the gaseous package components as a diluent for at least some of the conditioning components. Because the gaseous package components effectively dilute at least some of the conditioning components, the diluted conditioning components are easier to disperse in the delivered foam. While not wishing to be bound by theory, it is believed that the diluting effect of the gaseous package components causes the conditioning component to be present in the conditioning composition as significantly small droplets. As such, the composition may be characterized as a dispersion of the conditioning package components and the foaming package components in the aqueous liquid package components. This dispersion can be more easily generated into a foam upon being propelled from the container by the gaseous package components, but because the gaseous package components also dilute the conditioning components, the resulting, smaller droplets of the conditioning components are more easily and more evenly dispersed throughout the formed foam and are thus more easily applied evenly throughout dry hair.

Non-limiting examples of materials suitable for use in the gaseous package include butane, isobutane, propane, liquefied petroleum gas, dimethyl ether, trichlorofluoromethane, dichlorodifluoromethane, dichlorotetrafluorothane, monochlorodifluoromethane, trichlorotrifluoroethane propane, carbon dioxide, nitrous oxide, and combinations thereof. The components of the gaseous package preferably will rapidly evaporate from the conditioning composition during and after application of the foam to the dry hair. Left behind is an evenly applied amount of the fine droplets of the conditioning components that rapidly absorbs into the hair to cause the conditioning effects as further discussed herein. In some embodiments of the present disclosure, the gaseous package can comprise about 10% to about 30%, about 16% to about 24%, or about 18% to about 22% by weight of the conditioning composition based on the total weight of the conditioning composition. In certain embodiments, the gaseous package can comprise three different gaseous materials in fixed ratios to achieve the desired effects described herein. For example, a ratio of a first gaseous component, a second gaseous component, and a third gaseous component (relative to the weight of the gaseous package) can be such that the first gaseous component comprises about 51% to about 65% of the gaseous package and each of the second and third gaseous components independently comprises about 15% to about 30% by weight of the gaseous package. In some embodiments, the first gaseous component may comprise a major portion of the gaseous package (e.g., greater than 50%, greater than 55%, or greater than 60% by weight of the gaseous package, such as up to a maximum of about 90% by weight). As a non-limiting example of a combination of three gaseous materials, butane may be used as the first gaseous component, and isobutene and propane may be used as the second and third gaseous components.

A conditioning package useful according to the present disclosure preferably comprises a plurality of conditioning agents present in amounts effective to be retained by hair to which the conditioning composition is applied and to impart to the hair at least one conditioning effect. As used herein, a “conditioning effect” encompasses a temporary modification to the appearance, feel, and/or physical state of the hair to which the conditioning composition is applied. A conditioning effect can be specifically attributable to the presence of a specific conditioning agent, the presence of a combination of specific conditioning agents, and/or an amount of specific conditioning agent(s) present in the composition. Conditioning effects may also be attributable to the overall nature of the stable foam in which the conditioning composition is dispensed for use since such overall nature can be expressly configured to provide for the conditioning effect, such as by allowing for the use of conditioning agents that would not otherwise be expected to be useful for application to dry hair. Non-limiting examples of conditioning effects that may be provided to hair by application of the present conditioning composition include improving smoothness of the hair (i.e., a smoothing effect), hydrating the hair (e.g., a hydrating effect), increasing hair shine (i.e., a visual effect), reducing hair frizz (i.e., a structural effect), locking moisture into the hair (i.e., a hydrating effect), and providing a silky feel or touch to the hair (i.e., a structural effect). The conditioning effects likewise can be achieved in the absence of undesired effects. For example, conditioning effects can be provided by the conditioning composition without leaving the hair feeling heavy or weighed down and without leaving the hair feeling oily or greasy. Moreover, the conditioning effects can be rapidly achieved in light of the ability of the conditioning composition that is delivered as a stable foam being rapidly absorbed into the hair structure.

A benefit of the presently disclosed conditioning composition is the ability to apply the conditioning composition directly to dry hair and then leave it in place without rinsing away with water, wiping away with towels, brushing away with brushes, or the like. Rather, the conditioning composition is a leave-in composition, and the conditioning package agents are retained by the hair to which they are applied. By being retained by the hair, the conditioning agents are configured to provide the conditioning effects for a prolonged period of time without the undesired effects of heaviness, oiliness, greasiness, a wet look, or a stiff feeling that are often associated with leave-in conditioners. The effects can also be improved relative to rinse-away conditioners that are typically used during showering since the conditioner is only applied to the hair for a short time before being rinsed away with water. A vast majority of the conditioning agents in such compositions are rinsed away and cannot provide any prolonged effects to the hair. According to the present disclosure, however, the conditioning compositions are effective for conditioning the hair such that at least one conditioning effect imparted to the hair by the conditioning agents that are retained by the hair are likewise retained for an extended period of time. For example, the at least one conditioning effect imparted to the hair can be retained for at least 4 hours, at least 6 hours, at least 8 hours, at least 10 hours, or at least 12 hours after the conditioning composition is applied to the hair. In some embodiments, the conditioning effect may be retained by the hair until the hair is subjected to a later washing with a cleansing composition or rinsing with water.

In some embodiments, a conditioning agent useful in the conditioning package of the present disclosure can comprise one more conditioning agent effective for functioning as an emollient. An agent effective as an emollient can include materials that are effective at smoothing a surface to which they are applied, such as by filling gaps present at the surface or forming a thin coating layer on at least part of the surface. In the present disclosure, an emollient preferably leaves minimal or no coating layer and rather function to even out rough surfaces of hair by gap filling or otherwise smoothing out the hair surface. In particular, the outer cuticle layer of hair can be damaged by harsh chemical treatments, heat, friction (e.g., from brushing, styling, or rubbing against a pillow or headwear), or atmospheric elements (sunlight, wind, pollutants, etc.). Such damage often causes the individual cuticle cells to become raised instead of maintaining the natural, flattened, plate-like condition. Conditioning agents useful as emollients according to the present disclosure particularly can be effective as a smoothing component that causes raised hair cuticles to partially or completely return to the natural, flattened state. Emollients thus can be effective to provide the smoothing effect desired for hair but also can be effective to provide structural effects that are desired. For example, emollients can be effective to increase hair shine since the flattened cuticles are more effective reflectors of light than the raised cuticles, reduce hair frizz since the “frizziness” can arise at least in part from damaged and raised hair cuticles, and provide a silky feel or touch to the hair since the flattened cuticles impart such texture to the hair. In some embodiments, components effective as emollients may be characterized as occlusive agents in light of the ability to help lock moisture into the inner layers of the hair below the cuticle.

In certain embodiments, non-limiting examples of conditioning agents effective as emollients and/or effective to impart a smoothing effect and/or effective to impart a structural effect can include silicones (e.g., phenyl trimethicones, dimethicones, cyclomethicones, dimethicone copolyols, amino silicones, pegylated silicones, etc.), petroleum based cationic surfactants, distearyldimonium chloride, guar compounds including cationic polymers and guar gum, and polycationic compounds designated as polyquaternium 4, 6, 7, 10, or 22. In some embodiments, the emollient particularly is a hydrophobic emollient. For example, the emollient of the rinse-free shampoo composition can comprise a long chain alkane. As used herein, a long chain alkane is at least a C₁₀ alkane or at least a C₁₂ alkane, preferably up to a C₄₀ alkane. For example, in certain embodiments the rinse-free shampoo composition can comprise an emollient in the form of a C₁₃-C₁₅ alkane. In some embodiments, the emollient comprises a squalane and/or one or more derivatives thereof, particularly hemisqualane. In certain embodiments, the rinse-free shampoo composition can comprise PEG-8 dimethicone as an emollient and particularly a smoothing component effective to impart to hair a smoothing effect. Likewise, any of the foregoing may be effective for imparting to hair a smoothing effect and/or a structural effect that is evidence of conditioning of the hair by the conditioning composition.

In some embodiments, a conditioning agent useful in the conditioning package of the present disclosure can comprise one more conditioning agent effective for functioning as a humectant. An agent effective as a humectant can include materials that are effective at increasing natural moisture content of the hair and thus for functioning as a hydrating component effective to impart a hydrating effect to the hair. Natural moisture is contrasted with added surface water because added surface water imparts a wet, heavy, matted condition to the hair because of the wetting action of the water on the hair surface. Added surface water has little effect on hair moisture because the surface water is easily dried or evaporated away from the hair. Natural hair moisture, however, relates to moisture that is retained within the hair structure, such as held in, around, and below the individual hair cuticles. Natural hair moisture increases hair health, strength, and shape. Agents that increase natural moisture can be effective for providing a smoothing effect since hair with increased moisture content is more likely to have cuticles in the more natural, flattened state, for providing a hydrating effect, for increasing hair shine since hair with low moisture can appear dull, for providing a silky feel or touch to the hair since natural moisture can make hair feel softer, and for making hair more clastic and less brittle.

In certain embodiments, non-limiting examples of conditioning agents effective as humectants and/or effective to impart a hydrating effect and/or effective to impart a structural effect can include materials with reactive hydroxyl groups or similar groups that are effective for attracting molecular water to be retained as moisture within the hair structure. Useful humectants can be materials that are effective to increase absorption of water molecules into the hair or adsorption of water molecules onto the hair for further uptake. In some embodiments, polyols can be particularly useful as humectants and thus as a hydrating component of the conditioning package of the conditioning composition. In certain embodiments, glycerin in particular can be used as a humectant and thus as a hydrating component of the conditioning package. Further, non-limiting examples of humectants that may be useful include pantethol, hyaluronic acid, and aloe.

The conditioning package can comprise about 4% to about 12%, about 5% to about 10%, or about 6% to about 9% by weight of the present conditioning composition based on the total weight of the composition. In certain embodiments, a hydrating component of the conditioning package, such as a humectant, can comprise at least 2.5% by weight of conditioning composition, such as about 2.5% to about 5% by weight, based on the total weight of the composition. In certain embodiments, a smoothing component of the conditioning package, such as an emollient, can comprise at least 4% by weight of the conditioning composition, such as about 4% to about 7% by weight, based on the total weight of the composition.

A foaming package useful according to the present disclosure preferably comprises a plurality of components effective to provide the conditioning composition as a stable foam upon dispensing of the conditioning composition from a container. As discussed above, it is necessary for the conditioning composition to be dispensed as a stable foam so that the conditioning agents provided by the conditioning package are physically dispersed and separated by entrained gases in a manner that allows the conditioning agents to be substantially evenly applied to the hair as sufficiently small droplets so that the conditioning effects are achieved for substantially all of the hair and so that conditioning agents are not unevenly applied in undesirably large quantities to only small patches of hair. While the gaseous package helps in the dispersal of the conditioning agents, the foaming package includes required components to achieve the basic foam structure, to preserve the foam, and to stabilize the foam so that the foam does not prematurely collapse and lose the dispersal achieved with the gaseous package. Foam stability can be particularly important in light of the relatively high content of conditioning agents in the conditioning compositions that would be expected to cause the composition to be too heavy to form a foam that remains stable for a sufficiently long period of time.

Foaming agents useful in the foaming package of the present compositions can comprise one or more surfactants that are effective to reduce surface tension of the combined liquid components of the conditioning composition and thus improve case of foam generation by the propellant action of the gaseous package when the conditioning composition is dispensed from a container. Useful surfactants may particularly be surfactants that otherwise have low detergency properties. Likewise, the surfactants used as foaming agents may be used in concentrations that do not provide significant detergency properties. Non-limiting examples of surfactants useful as foaming agents can include materials in the cocamide family. For example, nonionic cocamide DEA, cocamidopropyl betaine, and cocamidopropyl hydroxysultainc. Foaming agents particularly may be provided in the form of a water soluble liquid. In some embodiments, a foaming agent used in the foaming package of the conditioning composition can comprise about 1% to about 4%, about 1.5% to about 3.5%, or about 2% to about 3% by weight based on the total weight of the conditioning composition.

Foam preservatives useful in the foaming package of the present compositions can comprise one or more materials that are effective against bacteria and thus are useful for maintaining the composition in a useful form for an extended period of time. Foam preservatives preferably can include materials that also provide at least some benefit in stabilizing the foam. Phenol ether compounds, such as phenoxyethanol, can be particularly useful. Preservatives can be present in the form of an oil and thus may provide some benefit in interacting with foaming agents in the form of water soluble liquids for forming contact points for the fatty portions of the foaming agent, such as the lauric acid groups present in cocamidopropyl betaine. In some embodiments, a foam preservative used in the foaming package of the conditioning composition can comprise about 0.01% to about 2%, about 0.1% to about 1.5%, or about 0.2% to about 1% by weight based on the total weight of the conditioning composition.

Foam stabilizers useful in the foaming package of the present compositions can comprise one or more materials that are effective to assist in preventing foam collapse in light of the presence of the relatively heavy conditioning components that are present in the conditioning composition in relatively large amounts. Although phenoxyethanol can provide stabilizing effects, it surprisingly has been found that foam stabilization can be achieved by the inclusion of particulate solids, such as starch solids. The particulate solids preferably are not substantially soluble in the aqueous liquid package components. Although not wishing to be bound by theory, it is believed that the presence of particulate solids provides solid structural elements that function essentially as anchor points in the foam structure as delivered from the container. In the absence of the particulate solids, the relative heaviness of the conditioning agents overcomes the balance between the liquid film network making up the non-gaseous elements of the foam and the pressure of the gases trapped within the “bubbles” or gas pockets that provide volume to the foam. When foam stability is tied solely to the foam former, resistance to collapse essentially entirely reliant on the strength of the liquid film network within the foam. A “stronger” liquid film network will have a denser structure that is more compact and thus makes is more difficult to evenly distribute the conditioning package throughout the hair. This makes for a difficult task in providing a foam with sufficient dispersal of conditioning agents to allow for high conditioning agent content, as already discussed above. The particulate solids are believed to allow for configuring the dispensed foam to have a high volume, which means more entrained gas and potentially a weaker liquid film network trapping the gas bubbles. The solids may provide anchor points within the liquid film network that increase structural integrity and prevent a cascading effect that is often seen with so-called collective bubble collapse in foams whereby breaking of bubbles near the outer surface of the foam mass cascades inward to rapidly disintegrate the foam. The solids thus can function essentially as an emulsifier between the oil, water, and gas phases in the stable foam. In testing carried out evaluating a foam according to the present disclosure and a foam that is otherwise identical except for the presence of the particulate solids, the samples without the particulate solids rapidly collapsed as the foam structure disintegrated. Foams according to the present disclosure, however, were found to be stable in that foam collapse was significantly slowed.

A stable foam, as used herein, is thus understood to mean a foam that significantly retains the majority of its initial volume present at the time of dispensing for a specific duration of time after dispensing. Foam stability is limited by the conditioning package in that the use of higher amounts of conditioning agents and/or the use of conditioning agents with higher unit weight will make it increasingly difficult to achieve a stable foam. The inclusion of a foam package as described herein, however, overcomes this problem and achieves a foam that is stable beyond what would typically be expected. In some embodiments, therefore, a stable foam may be characterized by the foam that is provided upon dispensing of the conditioning composition from a container having an initial volume, and that foam having a retained volume that is at least a certain percentage of the initial volume at a defined time after dispensing. Such characterization is highly relevant since a dispensed foam must retain its foam structure and its desired volume for a time sufficient to allow a user to thoroughly disperse the foam throughout the hair while avoid placement of the conditioning composition in only small portions of the hair. In some embodiments, a stable foam according to the present disclosure will retain at least 90% of its initial volume (i.e., volume at dispensing) at a time of 1 minute after dispensing, at a time of 3 minutes after dispensing, or at a time of 5 minutes after dispensing. In some embodiments, a stable foam according to the present disclosure will retain at least 80% of its initial volume (i.e., volume at dispensing) at a time of 1 minute after dispensing, at a time of 3 minutes after dispensing, or at a time of 5 minutes after dispensing. In some embodiments, a stable foam according to the present disclosure will retain at least 70% of its initial volume (i.e., volume at dispensing) at a time of 1 minute after dispensing, at a time of 3 minutes after dispensing, or at a time of 5 minutes after dispensing. In some embodiments, a stable foam according to the present disclosure will retain at least 60% of its initial volume (i.e., volume at dispensing) at a time of 1 minute after dispensing, at a time of 3 minutes after dispensing, or at a time of 5 minutes after dispensing. The stable foam thus resists disintegration or collapse or exhibits slowed disintegration or collapse by at least this quantifier.

As noted, particulate starch solids can be useful as the particulate solid used as a foam stabilizer in the foaming package of the present disclosure. Non-limiting examples of starch materials that can be useful in the present disclosure include corn starch, potato starch, tapioca starch, rice starch, wheat starch, cassaya starch, and combinations thereof. In certain embodiments, the rice starch in particular can be used. A starch material can be modified (e.g., through processes such as esterification, etherification, oxidation, acid hydrolysis, crosslinking, or enzyme conversion) or unmodified. Modified starch can be modified to improve resistance to solubilization in the aqueous liquid package components of the conditioning composition. In various embodiments, the rinse-free shampoo composition comprises distarch phosphate, and particularly distarch phosphate from rice starch. Distarch phosphate is an example of a starch that is modified, such as by crosslinking with sodium metaphosphate or, more particularly, sodium hexametaphosphate. In preferred embodiments, any of the starch materials noted above may be expressly excluded from the foam stabilizer, from the foaming package, and/or from the conditioning composition. As such, one specific starch type may be included to the exclusion of any or all of the other starch types noted.

The inclusion of particulate solids, and specifically particulate starch solids, can provide benefits in addition to slowing disintegration of the foam. In some embodiments, added particulate solids can be effective to increase smoothness of the foam. In some embodiments, added particulate solids can be effective to increase creaminess of the foam. Again, while not wishing to be bound by theory, it is believed that the presence of the particulate solids in the foam structure, particularly by inclusion within the liquid film network, is effective to increase the strength and resistance to breakage of the liquid film network. While this can provide slowing of disintegration of the foam, the added strength can likewise impart increased smoothness in that the bubbles within the foam structure are breaking and collapsing at a slower rate, and the sensation against the skin of a user is primarily the sensation of the texture of the liquid film network without intervening sensations of breakage of the liquid film network and release of the trapped gases. This effect combined with the increased amount and/or number of conditioning agents likewise can lead to the sensation of creaminess of the dispensed foam against the skin of a user.

The amount of particulate solids required to achieve the above properties as a foam stabilizer can be surprisingly low, particularly in light of the known uses of particulate starch solids in cleaning compositions intended for use in dry hair. For example, U.S. Patent Publication No. 2020/0390666 to Richardson, the disclosure of which is incorporated herein by reference, discloses rinse-free shampoo compositions that include starch in concentrations effective for removing oil and grease from the hair of a user. A relatively high content of the particulate starch is required in order to achieve the cleaning effect. Because the intended use of the present compositions is to impart conditioning effects to dry hair, however, it would be contrary to the intended use to include a material in a concentration that is known to remove oils from hair. Surprisingly, it has been found that particulate starch solids, and specifically particulate rice starch solids, can provide the above-described foam stabilizing effects when present in amounts that are below what would be effective for absorbing oil from hair and thus would be effective for cleaning hair or functioning essentially as a rinse-free shampoo for hair. As such, the foaming package can be characterized as including particulate starch in an amount that is substantially ineffective for absorbing oil from hair and/or that is substantially ineffective for causing the present compositions to function essentially as a rinse-free shampoo.

In some embodiments of the present disclosure, the conditioning compositions can include particulate solids generally, starch solids particularly, and rice starch solids even more particularly in an amount of no greater than 4.5% by weight, no greater than 4% by weight, no greater than 3.5% by weight, or no greater than 3% by weight, based on the total weight of the conditioning composition. In some embodiments, said solids can comprise about 0.1% to about 4.5% by weight, about 0.5% to about 4% by weight, about 1% to about 3.5% by weight, or about 1% to about 3% by weight based on the total weight of the conditioning composition.

The foaming package may be characterized in some embodiments in relation to the relative amounts of the foaming agent, the foam preservative, and the foam stabilizer. For example, the foaming package itself for use in the conditioning composition may comprise about 40% to about 60% by weight of foaming agent, about 1% to about 15% by weight of foam preservative, and about 30% to about 50% by weight of the foam stabilizer, based on the total weight of the foaming package. In some embodiments, the foaming package itself for use in the conditioning composition may comprise about 45% to about 55% by weight of foaming agent, about 8% to about 12% by weight of foam preservative, and about 35% to about 45% by weight of the foam stabilizer, based on the total weight of the foaming package.

The conditioning composition likewise may be characterized in some embodiments in relation to the relative amounts of the aqueous liquid package, the gaseous package, the conditioning package, and the foaming package. For example, the aqueous liquid package may comprise about 60% to about 75% by weight of the composition, the gaseous package may comprise about 16% to about 24% by weight of the composition, the conditioning package may comprise about 5% to about 10% by weight of the composition, and the foaming package may comprise about 3% to about 7% by weight of the composition, each of the foregoing being based on the total weight of the composition.

In various embodiments of the present disclosure, the rinse-free shampoo composition can comprise at least one fragrance. In some embodiments, a fragrance can be provided in the form of an essential oil. In certain embodiments, the weight percentage of one or more fragrances in the rinse-free shampoo composition, based on the total weight of the rinse-free shampoo composition, can be about 0.001% to about 3%, about 0.01% to about 2%, or about 0.1% to about 1%. In certain embodiments, the weight percentage of a fragrance in the rinse-free shampoo composition, based on the total weight of the rinse-free shampoo composition, can be at least about 0.001%, at least about 0.01%, at least about 0.1%, or at least about 1%, preferably with an upper limit of about 10% by weight.

In various embodiments, the conditioning composition can comprise one or more components in addition to the components of the aqueous liquid package, the gaseous package, the conditioning package, and the foaming package described above. For example, the conditioning composition can include at least one cooling agent (also referred to as a “refreshing agent”). In certain embodiments, the cooling agent can be in the form of an extract. For example, the rinse-free shampoo composition can include menthol. In some embodiments, the conditioning composition can include at least one fragrance, which may include essential oils, natural fragrances, and/or synthetically produced fragrances. In some embodiments, the conditioning composition can include at least one plant extract. For example, aloe barbadensis leaf extract may be used. Such additional components independently can be present in an amount of about 0.001% to about 3%, about 0.01% to about 2.5%, about 0.1% to about 2%, or about 0.5 to about 1.5% by weight based on the total weight of the conditioning composition.

In some embodiments, the conditioning composition may include one or more dyes. Since the conditioning composition is configured for application to dry hair, the composition can be particularly useful for application of temporary dye to hair. The temporary dye may be configured for providing temporary color to only a portion of an individual's hair or substantially all of the hair. In particular, the temporary dye may be provided in colors other than what are typically recognized to be “natural” hair colors (e.g., other than black, brown, blonde, or red). Thus use of dyes substantially corresponding to natural hair colors are not excluded, however, unless expressly stated. Temporary dyes in particular may be configured to impart colors such as pink, purple, blue, green, and variations thereof that are not commonly associated with a natural hair color. Dyes may be included in the conditioning composition in an amount of about 0.001% to about 3%, about 0.01% to about 2.5%, about 0.1% to about 2%, or about 0.5 to about 1.5% by weight based on the total weight of the conditioning composition.

The present disclosure further can relate to methods of preparing a conditioning composition that is configured for application as a foam to substantially dry hair. The methods can comprise combining the aqueous liquid package components. While stirring, the components of the foaming package are added with the exception of the particulate solid component. While stirring, the components of the conditioning package are added. Finally stirring, any additional components (e.g., fragrance, plant extracts, dye, etc.) are added. The completed mixture established a bulk liquid that can added in required amounts to dispensing containers. The method then comprises dosing the required amount of the particulate solids (e.g., starch solids, such as rice starch solids) into an empty dispensing container. The required amount of the bulk liquid is next added to the dispensing container. The dispensing container is then crimped closed with a valve, and the required content of the gaseous package components is added through the valve. Once all components are combined, the dispensing container is completed with addition of a dispensing actuator and cap.

Embodiments of the conditioning compositions described herein are configured such that, when dispensed from a container, the composition is in the form of a foam that can be applied to the substantially dry hair of a user. The applied composition can be distributed through the hair by massaging into the hair, for example, in order to substantially evenly distribute the conditioning agents to the hair and avoid localized, over application of conditioning agent. The conditioning composition preferably leaves little or no residue in the hair after application. The conditioning composition is also configured to have a substantially “lightweight” texture. This, in some embodiments, can be equated to a density of the dispensed foam. In some embodiments, the foam dispensed from the product container can have a density in the range of at least 0.3 g/cm³, at least 0.4 g/cm³, at least 0.5 g/cm³, or at least 0.6 g/cm³, such as up to a maximum of no more than 5 g/cm³, no more than 4 g/cm³, no more than 3 g/cm³, no more than 2 g/cm³, or no more than 1 g/cm³. In preferred embodiments, density can be about 0.4 g/cm³ to about 1 g/cm³, about 0.45 g/cm³ to about 0.9 g/cm³, or about 0.5 g/cm³ to about 0.8 g/cm³.

The conditioning composition provides a conditioning effect, is quick to use, does not leave any residue in the hair after use, and dries rapidly. For example, after completing application of the conditioning composition, the hair will have a feeling to the skin of being substantially dry (i.e., little or no external water remaining on hair surfaces) in a time of less than 1 minute, less than 2 minutes, less than 3 minutes, less than 4 minutes, or less than 5 minutes.

The present disclosure also can provide methods of conditioning hair. In the field of hair care, conditioning is typically done with compositions that are applied to wet hair and then rinsed away from the hair. Leave-in conditioners likewise are typically configured to be applied to wet hair. Moreover, leave-in conditioners leave hair looking wet, heavy, and/or oily until rinsed away with water. As such, methods of conditioning hair typically require application to wet hair.

Example methods of conditioning hair can comprise applying to substantially dry hair a conditioning composition as described herein. For example, the conditioning composition can comprise: an aqueous liquid package; a gaseous package effective as a propellant; a conditioning package comprising a plurality of conditioning agents present in amounts effective to be retained by hair to which the conditioning composition is applied and to impart to the hair at least one conditioning effect; and a foaming package comprising a plurality of components effective provide the conditioning composition as a stable foam upon dispensing of the conditioning composition from a container. In some embodiments, it can be desirable to combine conditioning of dry hair with cleansing of dry hair. The method of conditioning hair can be configured such that applying the conditioning composition is preceded by cleaning the hair with a dry shampoo composition.

The present methods of conditioning hair can be characterized in that applying the conditioning composition can be effective to impart to the hair the at least one conditioning effect in the substantial absence of providing any cleaning effect to the hair. In particular, the conditioning composition that is applied to the hair can exclude any component in an amount effective to be expected to impart a cleansing effect to the hair and/or expected to substantially remove oil from the hair.

### EXPERIMENTAL

### Example 1

A conditioning composition according to the present disclosure was subjectively evaluated by a group of 167 females, aged 18-34. The participants had a mix of hair types. All participants self-described as being interested in the product description and were willing to refrain from using their existing conditioner during the trial period. Table 1 provides a list of statements regarding product efficacy whereby participants were asked to rate product efficacy against pre-trial expectations using one of the following: much better than expected; slightly better than expected; about the same as expected; slightly worse than expected; and much worse than expected. The “% endorsement” in Table 1 is a sum of the responses of “much better than expected” and “slightly better than expected”.

TABLE 1

| Characterization of Product Efficacy | Endorsement (%) |
|---|---|
| Makes hair smoother | 88 |
| Makes hair look healthy | 86 |
| Makes hair feel softer | 86 |
| Locks in moisture | 84 |
| Hydrates hair | 84 |
| Revives hair | 83 |
| Gives a silky touch/silky finish | 83 |
| Adds shine to hair | 82 |
| Reduces fly aways | 82 |
| Doesn't weigh hair down | 81 |
| Makes hair feel healthy | 81 |
| De-tangles hair | 80 |
| Effect lasts all day | 80 |
| De-frizzes hair | 79 |
| Effect lasts 8 hours | 78 |
| Strengthens hair | 62 |

Table 2 provides a list of statements regarding product usage whereby participants were asked to rate their usage of the product against pre-trial expectations using one of the following: much better than expected; slightly better than expected; about the same as expected; slightly worse than expected; and much worse than expected. The “% endorsement” in Table 1 is a sum of the responses of “much better than expected” and “slightly better than expected”.

TABLE 2

| Characterization of Product Efficacy | Endorsement (%) |
|---|---|
| Is easy to dispense | 95 |
| Absorbs quickly into hair | 93 |
| Is convenient to use | 93 |
| Is light weight | 90 |
| Makes managing hair easier | 90 |
| Refreshes hair between washes | 89 |
| Fits into haircare routine | 87 |
| Does not leave hands sticky | 87 |
| Has an airy texture | 86 |
| Is something I would recommend | 86 |
| Is not messy | 86 |
| Makes hair more manageable | 85 |
| Does not leave hair greasy | 84 |
| Does not leave hair sticky | 83 |
| Leaves a residue in hair | 35 |

### Example 2

Testing was carried out to evaluate effect of including a particulate solid foam stabilizer. Test compositions were prepared so as to comprise 60% to 70% by weight of an aqueous liquid package, 15% to 25% by weight of a gaseous package, 5% to 10% by weight of a conditioning package, and 3% to 8% by weight of a foaming package. The aqueous liquid package, the gaseous package, and the conditioning package included components as otherwise described herein. The foaming package in each test sample included a foaming agent and a foam preservative. A first test formulation utilized 2% by weight of distarch phosphate as a foam stabilizer (particulate solid). A second test formulation included 1% by weight of distarch phosphate foam stabilizer. A third test formulation did not include any particulate solid foam stabilizer.

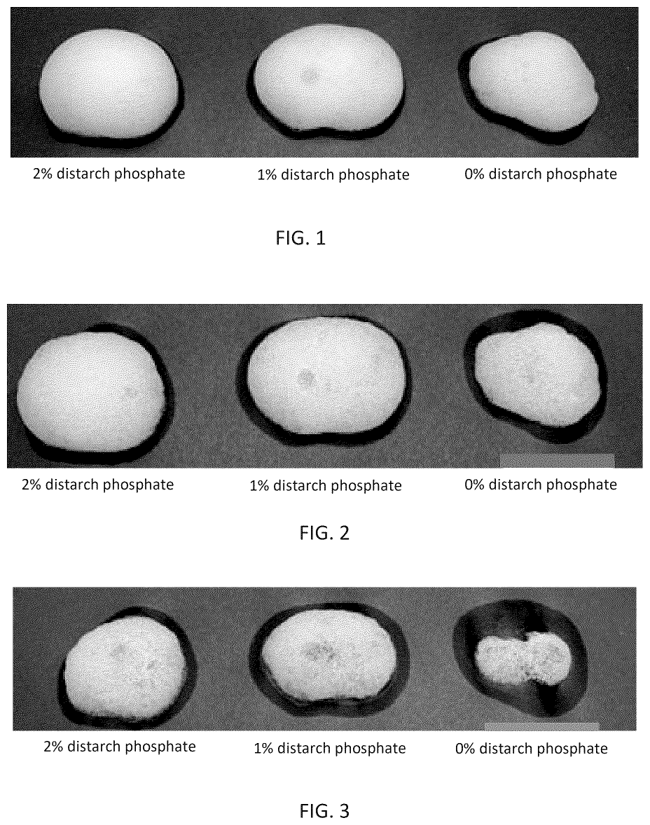

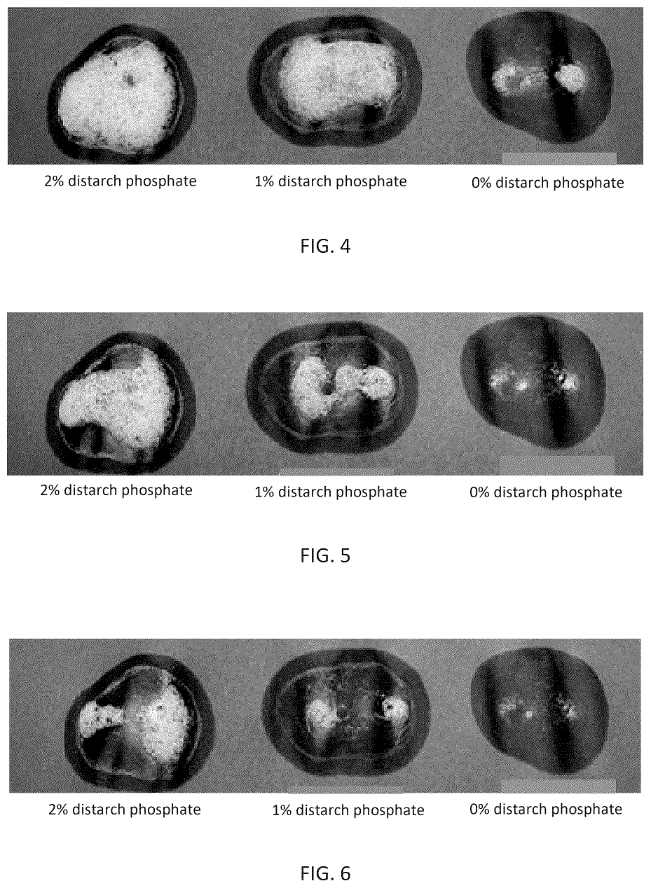

Each of the three test formulas was dispensed on black paper so that all three samples were dispensed within 30 second of each other. The initial foam produced is shown in FIG. 1 below. Follow-up photographs were taken at 3 minutes (FIG. 2), 6 minutes (FIG. 3), 9 minutes (FIG. 4), 12 minutes (FIG. 5), and 18 minutes (FIG. 6). In each photograph, the formulations are, from left to right, composition 1 (2% starch), composition 2 (1% starch), and composition 3 (no starch).

As can be seen in FIG. 1, the sample with 2% starch produced an initial foam with greater volume than either of the other samples and also with a more consistent texture and structure throughout the foam ball. The progression in each photograph shows the foam dissipation was greatest and fastest in the sample with no particulate starch solids. By six minutes after dispensing (FIG. 3), the sample with no starch solids is almost completely dissipated. Each sample including the distarch phosphate particulate solids dissipated slower than the no starch sample, and the sample with 2% starch provides superior results over the sample with 1% starch.

The terms “about” or “substantially” as used herein can indicate that certain recited values or conditions are intended to be read as encompassing the expressly recited value or condition and also values that are relatively close thereto or conditions that are recognized as being relatively close thereto. For example, unless otherwise indicated herein, a value of “about” a certain number or “substantially” a certain value can indicate the specific number or value as well as numbers or values that vary therefrom (+ or −) 2% or less, or 1% or less. Similarly, unless otherwise indicated herein, a condition that substantially exists can indicate the condition is met exactly as described or claimed or is within typical manufacturing tolerances or would appear to meet the required condition upon casual observation even if not perfectly meeting the required condition. In some embodiments, the values or conditions may be defined as being express and, as such, the term “about” or “substantially” (and thus the noted variances) may be excluded from the express value. Where a plurality of possible lower end values and a plurality of possible upper end values are provided for a particular parameter, it is understood that all possible combinations of values inclusive of any of the lower end values and any of the upper end values are encompassed for describing the parameter. Unless otherwise stated, it is understood that a listing of alternative material for a defined use includes the option to expressly exclude any one or more of the listed alternatives in any combination.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A conditioning composition comprising:
an aqueous liquid package;
a gaseous package effective as a propellant;
a conditioning package comprising a plurality of conditioning agents present in amounts effective to be retained by hair to which the conditioning composition is applied and to impart to the hair at least one conditioning effect; and
a foaming package comprising a plurality of components effective provide the conditioning composition as a stable foam upon dispensing of the conditioning composition from a container.

2. The conditioning composition of claim 1, wherein the aqueous liquid package comprises water and an alcohol present in a ratio of about 12 to 1 to a ratio of about 6 to 1 on a weight basis.

3. The conditioning composition of claim 1, wherein the plurality of conditioning agents in the conditioning package comprise at least a hydrating component effective to impart to the hair a hydrating effect and at least a smoothing component effective to impart to the hair a smoothing effect.

4. The conditioning composition of claim 3, wherein the hydrating component comprises a polyol, and the smoothing component comprises a silicone.

5. The conditioning composition of claim 4, wherein the polyol is present in an amount of at least 2.5% by weight based on the overall weight of the conditioning composition, and the silicone is present in an amount of at least 4% by weight based on the total weight of the composition.

6. The conditioning composition of claim 4, wherein the polyol is glycerin and the silicone is PEG-8 dimethicone.

7. The conditioning composition of claim 1, wherein the foaming package comprises each of a foaming agent, a foam preservative, and a foam stabilizer.

8. The conditioning composition of claim 7, wherein the foaming agent is a water soluble liquid, the foam preservative is an oil, and the foam stabilizer is a particulate solid.

9. The conditioning composition of claim 8, wherein the particulate solid foam stabilizer is effective to increase smoothness of the foam, increase creaminess of the foam, and slow disintegration of the foam.

10. The conditioning composition of claim 1, wherein the foaming package includes particulate starch in an amount substantially ineffective for absorbing oil from hair.

11. The conditioning composition of claim 10, wherein the particulate starch is rice starch present in an amount of no greater than 4.5% by weight based on the overall weight of the conditioning composition.

12. The conditioning composition of claim 1, wherein the aqueous liquid package comprises about 60% to about 75% by weight of the composition, the gaseous package comprises about 16% to about 24% by weight of the composition, the conditioning package comprises about 5% to about 10% by weight of the composition, and the foaming package comprises about 3% to about 7% by weight of the composition, each of the foregoing being based on the total weight of the composition.

13. The conditioning composition of claim 1, further comprising at least one of a fragrance, a plant extract, and a temporary dye.

14. The conditioning composition of claim 1, wherein the stable foam provided upon dispensing of the conditioning composition from container has a density of at least 0.3 g/cm³.

15. The conditioning composition of claim 1, wherein the stable foam provided upon dispensing of the conditioning composition from container has an initial volume, and wherein a retained volume of the stable foam at a time of 1 minute after dispensing is at least 90% of the initial volume.

16. The conditioning composition of claim 1, wherein the composition is effective for conditioning the hair such that the at least one conditioning effect imparted to the hair is retained for at least 8 hours after the conditioning composition is applied to the hair.

17. A method of conditioning hair, the method comprising applying to the hair when the hair is substantially dry a conditioning composition comprising:
an aqueous liquid package;
a gaseous package effective as a propellant;
a conditioning package comprising a plurality of conditioning agents present in amounts effective to be retained by hair to which the conditioning composition is applied and to impart to the hair at least one conditioning effect; and
a foaming package comprising a plurality of components effective provide the conditioning composition as a stable foam upon dispensing of the conditioning composition from a container.

18. The method of claim 17, wherein applying the conditioning composition is preceded by cleaning the hair with a dry shampoo composition.

19. The method of claim 17, wherein applying the conditioning composition is effective to impart to the hair the at least one conditioning effect in the substantial absence of providing any cleaning effect to the hair.
